(19) United States
(12) Patent Application Publication
(10) Pub. No.: US 2025/0161210 A1
(43) Pub. Date: May 22, 2025

(54) CHABATTE BEVERAGE COMPOSITION FOR ANTI-DIABETES AND METHOD FOR PREPARING THE SAME

(71) Applicant: Byeong In YOON, Gyeongsangnam-do (KR)

(72) Inventor: Byeong In YOON, Gyeongsangnam-do (KR)

(21) Appl. No.: 18/520,727

(22) Filed: Nov. 28, 2023

(30) Foreign Application Priority Data
Nov. 16, 2023 (KR) 10-2023-0158777

(51) Int. Cl. A61K 9/00; A61K 33/00; A61K 33/06; A61K 33/26; A61K 33/30; A61K 36/28; A61K 36/42; A61K 36/605; A61K 36/61; A61K 36/82

(52) U.S. Cl. CPC A61K 9/0095; A61K 33/00; A61K 33/06; A61K 33/26; A61K 33/30; A61K 36/28; A61K 36/42; A61K 36/605; A61K 36/61; A61K 36/82; A61K 2236/19

(57) ABSTRACT

An anti-diabetic CHABATAE beverage composition and a method for preparing the same are described, and the anti-diabetic CHABATAE beverage composition according to one embodiment includes mineral water; a fermented guava leaf extract; a fermented mulberry leaf extract; and an agarwood-fermented green tea extract.

### CROSS-REFERENCE TO RELATED APPLICATIONS

This application claims the priority of Korean Patent Application No. 10-2023-0158777 filed on Nov. 16, 2023, in the Korean Intellectual Property Office, the disclosure of which is incorporated herein by reference.

### BACKGROUND OF THE INVENTION

### Field of the Invention

The present invention relates to an anti-diabetic CHABATAE beverage composition having a high anti-diabetic activity and a method for preparing the same.

### Description of the Related Art

Diabetes is a chronic wasting disease characterized by a saccharometabolic dysfunction due to absolute deficiency of or resistance to insulin, and is a disease that causes various complications such as systemic lethargy, decreased resistance, vascular disorders, cerebral infarction, myocardial infarction, etc. Diabetes is largely divided into two types depending on the cause and pathological pattern thereof: insulin-dependent Type 1 diabetes, which is caused by the destruction of beta cells, insulin-secreting cells, due to the immunological mechanism of lymphocytes; and noninsulin-dependent Type 2 diabetes, which is caused by malfunctioning of insulin-secreting beta cells and increased resistance to insulin in peripheral target organs.

Type 1 diabetes mainly occurs in children and adolescents. Although the exact cause is not known, but it is known to be caused by genetic factors, environment, viruses, chemicals or the like. The symptoms are usually exhibited suddenly, and when the symptoms are severe, increased thirst, frequent urination, weight loss, extreme hunger, vomiting, abdominal pain, or fatigue may be exhibited. In Type 1 diabetes, the destruction of pancreatic beta cells results in insulin deletion, which ultimately disrupts the glucose homeostasis in the body. Since insulin plays a role of lowering the blood sugar level by transporting glucose in the blood into cells and converting glucose into glycogen in the liver, the lack of insulin due to the destruction of beta cells causes the destruction of blood glucose homeostasis and sustains hyperglycemia and metabolic disorders caused thereby. Therefore, insulin treatment is required in Type 1 diabetes.

Type 2 diabetes is characterized by insulin resistance in which insulin is relatively insufficient and the insulin function of lowering the blood sugar level is decreased and so the cells are unable to burn glucose effectively. Type 2 diabetes accounts for more than 90% of all diabetes, and recently, as the number of obese people increases due to the inactive lifestyle and the Westernized diet, Type 2 diabetes is rapidly increasing. The most effective treatment Type 2 diabetes is to achieve an optimal blood sugar level after a meal, and delayed absorption of monosaccharides through the inhibition of α-glucosidase activity is suggested as a beneficial strategy for treating Type 2 diabetes.

Thiazolidinediones, sulfonylureas, biguanides, benzoic acid derivatives, α-glucosidase inhibitors and others are used in the treatment of diabetes. However, since the treatment of diabetes using these drugs causes many side effects, the World Health Organization (WHO) strongly recommends the use of natural substances with fewer side effects against diabetes.

### PRIOR ARTS

[Patent Document] Korea Patent Publication No. 10-2020-0144857

### SUMMARY OF THE INVENTION

The technical problem to be solved by the present invention is to provide an anti-diabetic Chabatte beverage composition with an enhanced anti-diabetic activity and a method for preparing the same.

The technical problem to be solved by the present invention is not limited to the technical problem mentioned above, and other technical problems that are not mentioned can be clearly understood by those skilled in the art from the description below.

To achieve the technical problem above, one embodiment of the present invention provides an anti-diabetic CHABATAE beverage composition including mineral water; a fermented guava leaf extract; a fermented mulberry leaf extract; and an agarwood-fermented green tea extract.

In an embodiment of the present invention, the mineral water may include at least one selected from the group consisting of calcium, potassium, germanium, magnesium, iron, and zinc.

In an embodiment of the present invention, the mineral water may have a pH of 7 to 11.

In an embodiment of the present invention, the mineral water may be 95% to 98% by volume of the anti-diabetic CHABATAE beverage composition.

In an embodiment of the present invention, the fermented guava leaf extract and the fermented mulberry leaf extract may be 0.5% to 3% by volume in the anti-diabetic CHABATAE beverage composition.

In an embodiment of the present invention, the fermented guava leaf extract may be made from early-maturing guava leaves.

In an embodiment of the present invention, the agarwood-fermented green tea extract may be 0.5% to 3% by volume in the anti-diabetic CHABATAE beverage composition.

In an embodiment of the present invention, the composition may further include a Jerusalem artichoke-bitter gourd extract, wherein the Jerusalem artichoke-bitter gourd extract may be 0.5% to 3% by volume in the anti-diabetic CHABATAE beverage composition.

Another embodiment of the present invention provides a method for preparing an anti-diabetic CHABATAE beverage composition, the method including: a step of preparing fermented mulberry leaves and fermented guava leaves by fermenting mulberry leaves and guava leaves; a step of obtaining a fermented mulberry leaf-fermented guava leaf extract by putting the fermented mulberry leaves, the fermented guava leaves, and mineral water into a non-pressurized extractor and heating the same; a step of obtaining an agarwood-fermented green tea extract by putting mineral water and agarwood-fermented green tea into a non-pressurized extractor and heating the same; and a step of obtaining a mixed extract by putting the fermented mulberry leaf-fermented guava leaf extract and the agarwood-fermented green tea extract into heated mineral water and heating the same.

In an embodiment of the present invention, the step of preparing fermented mulberry leaves and fermented guava leaves may be fermenting the mulberry leaves and the guava leaves in a red clay fermentation reactor for 20 to 48 hours at a humidity of 70% to 80% and a temperature of 50° C. to 90° C.

In an embodiment of the present invention, the step of obtaining a fermented mulberry leaf-fermented guava leaf extract may be putting 90% to 95% by weight of the mineral water, 1% to 4% by weight of the fermented mulberry leaves, and 2% to 6% by weight of the fermented guava leaves to a non-pressurized extractor and extracting for 1 to 12 hours in a temperature range of 60° C. to 100° C.

In an embodiment of the present invention, the step of obtaining an agarwood-fermented green tea extract may be putting 95% to 98% by weight of the mineral water and 2% to 5% by weight of the agarwood-fermented green tea into the non-pressurized extractor and extracting for 1 to 12 hours in a temperature range of 60° C. to 100° C.

In an embodiment of the present invention, the step of obtaining a mixed extract may be putting 95% to 98% by volume of the heated mineral water, 0.5% to 3% by volume of the fermented mulberry leaf-fermented guava leaf extract, and 0.5% to 3% by volume of the agarwood-fermented green tea extract into the non-pressurized extractor and extracting for 1 to 12 hours in a temperature range of 80° C. to 120° C.

In an embodiment of the present invention, the step of obtaining the mixed extract may further include a Jerusalem artichoke-bitter gourd extract.

In an embodiment of the present invention, the Jerusalem artichoke-bitter gourd extract may be obtained by putting mineral water, Jerusalem artichoke, and bitter gourd into a non-pressurized extractor and heating the same.

In an embodiment of the present invention, the Jerusalem artichoke-bitter gourd extract may be obtained by putting 90% to 95% by weight of the mineral water, 1% to 6% by weight of the Jerusalem artichoke, and 1% to 4% by weight of the bitter gourd into a non-pressurized extractor and extracting for 1 to 12 hours in a temperature range of 60° C. to 100° C.

In an embodiment of the present invention, the method may include, after the step of obtaining a mixed extract, a step of sterilizing the mixed extract for 5 to 60 minutes in a temperature range of 90° C. to 130° C.

According to an anti-diabetic CHABATAE beverage composition according to an embodiment of the present invention, a beverage which has an excellent flavor with improved palatability, intake, and convenience by including fermented guava leaves, fermented mulberry leaves, and agarwood-fermented green tea, which is convenient to intake or drink every day, and which has an improved anti-diabetic activity may be provided.

According to a method for preparing an anti-diabetic CHABATAE beverage composition according to one embodiment of the present invention, a fermented guava leaf-fermented mulberry leaf extract and an agarwood-fermented green tea extract may be individually prepared and manufactured to be provided as a beverage composition for preventing, treating, and ameliorating diabetes with an improved flavor.

The effects of the present invention are not limited to the above-described effects, and should be understood as including all effects that can be inferred from the configuration of the invention described in the detailed description or claims of the present invention.

### BRIEF DESCRIPTION OF THE DRAWINGS

FIG. 1 is a flow chart representing the method for preparing an anti-diabetic CHABATAE beverage composition according to one embodiment of the present invention.

### DETAILED DESCRIPTION OF THE EMBODIMENTS

Hereinafter, the present invention will be described with reference to the attached drawings. However, the present invention may be implemented in various different forms and, therefore, is not limited to the embodiments described herein. In addition, to clearly explain the present invention in the drawings, parts that are not related to the description are omitted, and similar parts are given similar reference numerals throughout the specification.

Throughout the specification, when a part is described to be “connected (conjugated, contacted, combined)” with another part, this means not only a case of being “directly connected” but also a case of being “indirectly connected” with yet another member therebetween. In addition, when a part is described to “include” a certain component, this does not mean that other components are excluded, but that other components may be further included, unless specifically described otherwise.

The terms used in the present specification are only used to describe specific embodiments and are not intended to limit the invention. Singular expressions include plural expressions unless the context clearly dictates otherwise. In the present specification, terms such as “include” or “have” are intended to designate the presence of features, numbers, steps, operations, components, parts, or combinations thereof described in the specification and should be understood as not excluding in advance the presence or the possibility of adding one or more other features or numbers, steps, operations, components, parts, or combinations thereof.

Hereinafter, embodiments of the present invention will be described in detail with reference to the attached drawings.

An anti-diabetic CHABATAE beverage composition according to one embodiment of the present invention includes: mineral water; a fermented guava leaf extract; a fermented mulberry leaf extract; and an agarwood-fermented green tea extract.

In an embodiment of the present invention, the mineral water may include at least one selected from the group consisting of calcium, potassium, germanium, magnesium, iron, and zinc.

The mineral water, also referred to as spa water, is water containing a trace of mineral components such as calcium, magnesium, and potassium. Minerals account for 4% of the components of the human body, but since they are not produced in the body, they must be taken in through water or food. The present invention includes mineral water so that mineral components that are abundant in human body may be taken in.

As a method for producing the mineral water, for example, deep ocean water may be desalinated to produce mineral water. At this time, mineral water including mineral components in an amount of 0.01% by weight to 0.09% by weight is used to ensure that minerals that are lacking in the human body may be sufficiently taken in.

In an embodiment of the present invention, the mineral water may have a pH of 7 to 11; 7 to 10; 7 to 9; 7 to 8; 8 to 11; 8 to 10; 8 to 9; 8 to 11; 8 to 10; 8 to 9; 9 to 11; 9 to 10; or 10 to 11.

In an embodiment of the present invention, the mineral water may be 95% to 98% by volume; 95% to 97% by volume; 95% to 96% by volume; 96% to 98% by volume; 96% to 97% by volume; or 97% to 98% by volume of the anti-diabetic CHABATAE beverage composition.

Guava is a mysterious plant that is native to the tropical regions of the Central and South Americas from southern Mexico to Peru, and all of its leaves, roots, and fruits can be consumed. In particular, guava leaves contain antihistamines, which prevent rhinitis and have high antioxidant power, which is effective in removing free radicals secreted by inflammatory cells. In addition, since it contains a large amount of polyphenol compounds, it inhibits the mechanism of decomposing carbohydrates into glucose, ultimately having a significant effect in preventing diabetes through the effect of inhibiting an increase of the blood sugar level. In addition to polyphenol compounds, guava leaves also contain vitamin C and lycopene, which is contained in tomatoes in a large amount, thereby reducing the incidence rate of cardiovascular diseases such as high blood pressure, hyperlipidemia, and arteriosclerosis. Among polyphenol compounds, catechin decomposes body fat in the body, and thus guava is an excellent plant that is effective in a diet.

Mulberry is an arboreal deciduous tree that belongs to the Urticales order of angiosperms, the Moraceae family, and the Moraceae genus, and is widely distributed from tropical regions to temperate regions. In Korea, there are 13 varieties of mulberry trees in 5 genera. In the past, mulberry leaves were mainly used as food for silkworms, but many of the excellent effects of mulberry leaves have been revealed, and they are currently used as edible/pharmaceutical materials. Studies have shown that the main components of mulberry leaves are alanine, aspartic acid, and glutamic acid, which are amino acids contained in a high content. In particular, mulberry leaves are helpful in vascular health, have an antioxidant effect, make blood clear and make blood circulation smooth, are helpful in preventing vascular diseases and adult diseases, and prevent thrombosis.

In an embodiment of the present invention, the fermented guava leaf extract and the fermented mulberry leaf extract may be 0.5% to 3% by volume; 0.5% to 2% by volume; 0.5% to 1% by volume; 1% to 3% by volume; 1% to 2% by volume; 1% to 3% by volume; or 1% to 2% by volume of the anti-diabetic CHABATAE beverage composition.

In an embodiment of the present invention, the fermented guava leaf extract may be made from early-maturing guava leaves.

When the early-maturing guava leaves are used, the scent is better and the astringent taste is less than late-maturing guava leaves.

In an embodiment of the present invention, the agarwood fermented green tea extract may be 0.5% to 3% by volume; 0.5% to 2% by volume; 0.5% to 1% by volume; 1% to 3% by volume; 1% to 2% by volume; or 2% to 3% by volume of the anti-diabetic CHABATAE beverage composition.

Agarwood, which belongs to the thymelaeaceous family and has an scientific name of Aquilaria agallocha Roxburgh, is a stem tree with deposited resin. Agarwood, also referred to as aloeswood, means the part where the resin naturally secreted from agarwood is deposited and forms a histologically solid lump in the heartwood region of the agarwood. In herbal medicine, it is recorded that ‘agarwood lowers the energy and warms up the inside of the body.’ Together with ambergris and musk that are used for Gong Jin Dang, agarwood has been counted as one of the three major perfumes. The nutritional components of agarwood include benzylacetone, methoxybenzyl, alphabullezene, deraguaniene, currbitacin, and agaropyrol, and have the effect of relaxing the mind and strengthening the five intestines and liver.

According to an anti-diabetic CHABATAE beverage composition according to an embodiment of the present invention, a beverage which has an excellent flavor with improved palatability, intake, and convenience by including fermented guava leaves, fermented mulberry leaves, and agarwood-fermented green tea, which is convenient to intake or drink every day, and which has an improved anti-diabetic activity may be provided.

Another embodiment of the present invention provides a method for preparing an anti-diabetic CHABATAE beverage composition, the method including: a step of preparing fermented mulberry leaves and fermented guava leaves by fermenting mulberry leaves and guava leaves; a step of obtaining a fermented mulberry leaf-fermented guava leaf extract by putting the fermented mulberry leaves, the fermented guava leaves, and mineral water into a non-pressurized extractor and heating the same; a step of obtaining an agarwood-fermented green tea extract by putting mineral water and agarwood-fermented green tea into a non-pressurized extractor and heating the same; and a step of obtaining a mixed extract by putting the fermented mulberry leaf-fermented guava leaf extract and the agarwood-fermented green tea extract into heated mineral water and heating the same.

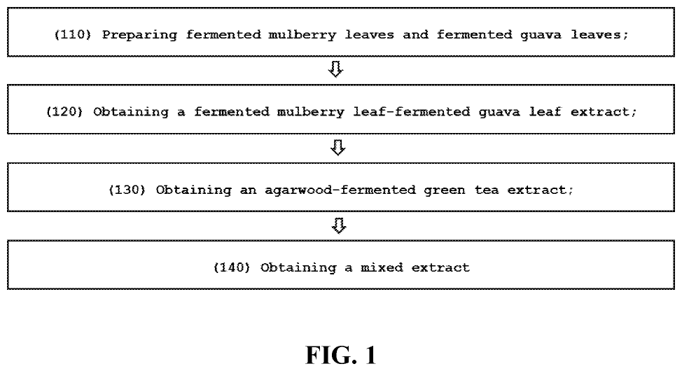

FIG. 1 is a flow chart representing the method for preparing an anti-diabetic CHABATAE beverage composition according to one embodiment of the present invention.

Referring to FIG. 1, the method for preparing an anti-diabetic CHABATAE beverage composition includes: a step of preparing fermented mulberry leaves and fermented guava leaves 110; a step of obtaining a fermented mulberry leaf-fermented guava leaf extract 120; a step of obtaining an agarwood-fermented green tea extract 130; and a step of obtaining a mixed extract 140.

The step of preparing fermented mulberry leaves and fermented guava leaves 110 is fermenting mulberry leaves and guava leaves.

In an embodiment of the present invention, the step of preparing fermented mulberry leaves and fermented guava leaves may be fermenting the mulberry leaves and the guava leaves in a red clay fermentation reactor for 20 to 48 hours at a humidity of 70% to 80% and a temperature of 50° C. to 90° C.

The red clay fermentation reactor may be made of red clay marble. Red clay has the mineralogical properties of clay minerals such as plasticity, adsorption, absorption and dehydration, suspension, and ion exchange. Red clay has a higher activity than other minerals, and is a natural material with various properties such as catalytic activity, high surface area, and absorption and emission of electromagnetic waves. When such red clay is used, since it has excellent soil humidity regulating capability and contains calcium carbonate to neutralize acid rain, an effect of preventing the acidification of soil may be obtained. Furthermore, red clay has an excellent ability to regulate its own humidity and is a very beneficial material for the human body since it emits far-infrared rays. Effects such as improving water quality and soil quality through the actions of various inorganics and enzymes are known, and they very environment-friendly components. Therefore, fermented mulberry leaves and fermented guava leaves that are fermented in a red clay fermentation reactor can impart an excellent flavor.

The fermentation may be performed in a humidity range of 70% to 80%; 70% to 75%; or 75% to 80%, and a temperature range of 50° C. to 90° C.; 50° C. to 80° C.; 50° C. to 70° C.; 50° C. to 60° C.; 60° C. to 90° C.; 60° C. to 80° C.; 60° C. to 70° C.; 70° C. to 90° C.; 70° C. to 80° C.; or 80° C. to 90° C. for 20 hours to 48 hours; 20 to 36 hours; or 36 hours to 48 hours.

When the conditions are out of the humidity range, fermentation temperature range, and process time described above, there is a risk that the astringent taste may become stronger.

In the step 120 of obtaining the fermented mulberry leaf-fermented guava leaf extract, the fermented mulberry leaf, the fermented guava leaf, and mineral water are placed in a non-pressure extractor and heated to obtain the fermented mulberry leaf-fermented guava leaf extract.

The step of obtaining a fermented mulberry leaf-fermented guava leaf extract 120 is obtaining a fermented mulberry leaf-fermented guava leaf extract by putting the fermented mulberry leaves, the fermented guava leaves, and mineral water into a non-pressure extractor and heating the same.

The above amount of the fermented mulberry leaves and the fermented guava leaves refers to a dry weight thereof.

In an embodiment of the present invention, the step of obtaining a fermented mulberry leaf-fermented guava leaf extract may be putting 90% to 95% by weight of the mineral water, 1% to 4% by weight of the fermented mulberry leaves, and 2% to 6% by weight of the fermented guava leaves to the non-pressurized extractor and extracting for 1 to 12 hours in a temperature range of 60° C. to 100° C.

The mineral water may include at least one selected from the group consisting of calcium, potassium, germanium, magnesium, iron, and zinc.

The mineral water may be 90% to 95% by weight; 90% to 94% by weight; 90% to 93% by weight; 90% to 92% by weight; 90% to 91% by weight; 91% to 95% by weight; 91% to 94% by weight; 91% to 93% by weight; 91% to 92% by weight; 92% to 95% by weight; 92% to 94% by weight; 92% to 93% by weight; 93% to 95% by weight; or 94% to 95% by weight.

The fermented mulberry leaves may be 1% to 4% by weight; 1% to 3% by weight; 1% to 2% by weight; 2% to 4% by weight; 2% to 3% by weight; or 3% to 4% by weight.

The fermented guava leaves may be 2% to 6% by weight; 2% to 5% by weight; 2% to 4% by weight; 2% to 3% by weight; 3% to 6% by weight; 3% to 5% by weight; 3% to 4% by weight; 4% to 6% by weight; 4% to 5% by weight; or 5% to 6% by weight.

The extraction may be performed with a non-pressure extractor in a temperature range of 60° C. to 100° C.; 60° C. to 90° C.; 60° C. to 80° C.; 60° C. to 70° C.; 70° C. to 100° C.; 70° C. to 90° C.; 70° C. to 80° C.; 80° C. to 100° C.; 80° C. to 90° C.; or 90 C to 100° C. for 1 hour to 12 hours; 1 hour to 10 hours; 1 hour to 8 hours; 1 hour to 5 hours; 3 hours to 12 hours; 3 hours to 10 hours; 3 hours to 8 hours; 3 hours to 5 hours; 5 hours to 12 hours; 5 hours to 10 hours; 5 hours to 8 hours; or 8 hours to 12 hours.

The extraction temperature and time are not particularly limited, but it is natural that the temperature and time should be such that sufficient extraction is possible without destroying the active ingredients in fermented mulberry leaves and the fermented guava leaves.

The extraction temperature and time are not particularly limited, but it is obvious that the temperature and time should be such that sufficient extraction is possible without destroying the active ingredients in the fermented mulberry leaves and fermented guava leaves.

The step of obtaining an agarwood-fermented green tea extract 130 is obtaining an agarwood-fermented green tea extract by putting mineral water and agarwood-fermented green tea into a non-pressurized extractor and heating the same.

First, the method for preparing the agarwood-fermented green tea includes: a drying step of drying the agarwood and tea leaves in the shade at room temperature for 5 to 7 hours; a step of roasting the tea leaves that have undergone the drying step, in a roaster, wherein 15 g to 30 g of agarwood pulverized into powder particles of 250 mesh to 350 meshes is added per 3 kg of tea leaves and the resulting mixture is continuously roasted for 10 to 20 minutes; a fermenting step of putting the tea leaves mixed with agarwood through the roasting step into a pine container and storing the same at a temperature of 30° C. to 40° C. for 5 to 7 hours; and a pressurizing step of steaming for 30 seconds the tea leaves that have undergone the fermenting step and then drying the same again.

In an embodiment of the present invention, the step of obtaining an agarwood-fermented green tea extract may be putting 95% to 98% by weight of the mineral water and 2% to 5% by weight of the agarwood-fermented green tea into the non-pressurized extractor and extracting for 1 to 12 hours in a temperature range of 60° C. to 100° C.

The above amount of the agarwood-fermented green tea refers to a dry weight thereof.

The extraction temperature and time are not particularly limited, but it is obvious that the temperature and time should be such that sufficient extraction is possible without destroying the active ingredients in the agarwood-fermented green tea extract.

The step of obtaining a mixed extract 140 is obtaining a mixed extract by putting the fermented mulberry leaf-fermented guava leaf extract and the agarwood-fermented green tea extract into heated mineral water and heating the same.

The mineral water may be heated at a temperature of 100° C.

In an embodiment of the present invention, the step of obtaining a mixed extract may be putting 95% to 98% by volume of the heated mineral water, 0.5% to 3% by volume of the fermented mulberry leaf-fermented guava leaf extract, and 0.5% to 3% by volume of the agarwood-fermented green tea extract into the non-pressurized extractor and extracting for 1 to 12 hours in a temperature range of 80° C. to 120° C.

The mineral water may be 95% to 98% by volume; 95% to 97% by volume; 95% to 96% by volume; 96% to 98% by volume; 96% to 97% by volume; or 97% to 98% by volume.

The fermented mulberry leaf-fermented guava leaf extract may be 0.5% to 3% by volume; 0.5% to 2% by volume; 0.5% to 1% by volume; 1% to 3% by volume; 1% to 2% by volume; 1% to 3% by volume; or 1% to 2% by volume.

When the fermented mulberry leaf-fermented guava leaf extract is less than 0.5% by volume, the elution amount of active ingredients such as polyphenols may be insufficient, and when the amount thereof is more than 3% by volume, it is ineffective because the cost of increasing the amount of raw materials used is increased more, compared to the increase of the amount of active ingredients such as polyphenols.

The agarwood-fermented green tea extract may be 0.5% to 3% by volume; 0.5% to 2% by volume; 0.5% to 1% by volume; 1% to 3% by volume; 1% to 2% by volume; or 2% to 3% by volume.

When the agarwood-fermented green tea extract is less than 0.5% by volume, the anti-diabetic activity and the elution amount of anti-diabetic active ingredients may be insufficient, and when the amount thereof is more than 3% by volume, it is ineffective because the cost of increasing the amount of raw materials used is increased more, compared to the increase of the amount of active ingredients.

In an embodiment of the present invention, the step of obtaining the mixed extract may further include a Jerusalem artichoke-bitter gourd extract.

In an embodiment of the present invention, the Jerusalem artichoke-bitter gourd extract may be obtained by putting mineral water, Jerusalem artichoke, and bitter gourd into a non-pressurized extractor and heating the same.

In an embodiment of the present invention, the Jerusalem artichoke-bitter gourd extract may be obtained by putting 90% to 95% by weight of the mineral water, 1% to 6% by weight of the Jerusalem artichoke, and 1% to 4% by weight of the bitter gourd into a non-pressurized extractor and extracting for 1 to 12 hours in a temperature range of 60° C. to 100° C.

When the Jerusalem artichoke-bitter gourd extract is further included, the Jerusalem artichoke-bitter gourd extract may be 0.5% to 3% by volume in the final anti-diabetic CHABATAE beverage composition.

The above amount of the Jerusalem artichoke and the bitter gourd refers to a dry weight thereof.

In an embodiment of the present invention, the method may include, after the step of obtaining a mixed extract, a step of sterilizing the mixed extract for 5 to 60 minutes in a temperature range of 90° C. to 130° C.

When the sterilization temperature is less than 90° C., sterilization may not be performed sufficiently, and when it exceeds 130° C., there is a risk that the active ingredients in the contents may be destroyed or deteriorated.

However, when the mixed extract is in a heated state, the sterilizing step may be omitted by bottling in a heated state.

Finally, the mixed extract may be filtered and bottled to finally prepare an anti-diabetic CHABATAE beverage composition.

According to a method for preparing an anti-diabetic CHABATAE beverage composition according to one embodiment of the present invention, a fermented guava leaf-fermented mulberry leaf extract and an agarwood-fermented green tea extract may be individually prepared and manufactured to be provided as a beverage composition for preventing, treating, and ameliorating diabetes with an improved flavor.

Hereinafter, the present invention will be described in detail with reference to the following examples and comparative examples. However, the technical idea of the present invention is not limited or restricted thereby.

### EXAMPLE

### Preparation of Fermented Mulberry Leaves and Fermented Guava Leaves

Mulberry leaves and early-maturing guava leaves were fermented in a fermentation reactor made of red clay marble for 24 hours at a humidity of 75% and a temperature of 60° C. to prepare fermented mulberry leaves and early-maturing guava leaves.

### Extraction of a Fermented Mulberry Leaf-Fermented Guava Leaf Extract

708 g of the fermented mulberry leaves and 1,416 g of the guava leaves were put into 28.32 L of mineral water in a non-pressure extractor and heated at 85° C. for 4 hours, and an fermented mulberry leaf-fermented guava leaf extract was extracted by using a non-pressure extractor.

### Preparation of an Agarwood-Fermented Green Tea

After drying the agarwood and tea leaves in the shade at room temperature for 5 to 7 hours, they were put into a roaster. 20 g of agarwood pulverized into powder particles of 250 mesh to 350 meshes was added per 3 kg of tea leaves and blended to be harmonious while roasting for 15 minutes. Afterwards, the resulting mixture was put into a pine container, underwent a fermenting step where it was stored at 35° C. for 6 hours, and then steamed for 30 seconds and dried again.

### Extraction of an Agarwood Fermented Green Tea Extract

2,124 g of the agarwood-fermented green tea was put into 28.32 L of mineral water in a non-pressurized extractor and fermented at 85° C. for 4 hours to extract a fermented agarwood-fermented green tea extract by using a non-pressure extractor.

### Preparation of an Anti-Diabetic CHABATAE Beverage Composition

30 L of the fermented mulberry leaf-fermented guava leaf extract and 33 L of the agarwood-fermented green tea extract were added to 1,800 L of mineral water heated to 100° C., and the resulting mixture was heated and filtered by using a non-woven filter under reduced pressure to prepare an anti-diabetic CHABATAE beverage composition.

### Experimental Example 1: DDPH and ABTS Radical Scavenging Activity

### Evaluation of Antioxidant Activity of the Anti-Diabetic Material Extracts

### <DPPH Radical Scavenging Activity>

DPPH assay measurement was performed in a 96-well plate by adding 100 μL of a solvent in which the extract was extracted for the negative control group and 100 μL of the sample for the test group, and 50 μL of 0.5 mM 1,1-diphenyl-2-picrylhydrazyl (DPPH-ethanol) solution was added and mixed. After the resulting mixture was allowed to react at 25° C. for 30 minutes, the absorbance was measured at 517 nm. The radical scavenging capacity of the extract relative to the control group in which only the solvent was added was expressed as a percentage.

### <ABTS Radical Scavenging Activity>

After mixing 5 ml of 7 mM ABTS and 88 μl of 140 mM potassium persulfate, the mixture was blocked from light for 16 hours at room temperature to form ABTS cations. Afterwards, this solution was diluted with PBS to obtain an absorbance value of 1.5 at 414 nm, 190 μL of the prepared diluted solution was mixed with 10 μL of the sample. The resulting mixture was allowed to react at room temperature for 6 minutes, and the absorbance was measured at 734 nm.

The radical scavenging activity was calculated as follows and expressed as a percentage.

$$\frac{DPPH}{ABTS} ( Radical scavenging activity ( \% ) ) = \frac{Control group absorbance - Absorbance with added sample}{Control group absorbance} \times 100$$

The DPPH radical scavenging activity of the anti-diabetic CHABATAE beverage composition of the present invention was compared with that of the 100% agarwood-fermented green tea extract (Comparative Example).

Table 1 below shows the DPPH radical scavenging activity (%), and Table 2 below shows the ABTS radical scavenging activity (%).

TABLE 1

| Concentration | Comparative | |
|---|---|---|
| (%) | Example | Example |
| 10 | 19.3 ± 0.8 | 25.3 ± 1.2 |
| 20 | 24.2 ± 0.7 | 26.8 ± 4.7 |
| 50 | 32.7 ± 0.2 | 41.2 ± 1.1 |
| 100 | 41.1 ± 1.1 | 53.1 ± 2.7 |

TABLE 2

| Concentration | Comparative | |
|---|---|---|
| (%) | Example | Example |
| 1 | 23.6 ± 0.9 | 72.4 ± 0.6 |
| 2 | 36.8 ± 0.5 | 98.8 ± 0.5 |
| 5 | 87.8 ± 2.5 | 98.7 ± 0.5 |
| 10 | 99.3 ± 0.5 | 99.2 ± 0.4 |

It was confirmed that the DPPH and ABTS radical scavenging activity was excellent at a low concentration (10%) and a high concentration (100%) compared to the 100% agarwood-fermented green tea extract.

### Experimental Example 2: Evaluation of Anti-Diabetic Activity of the Anti-Diabetic Material Extract

### <α-Glucosidase Inhibitory Activity>

For α-Glucosidase inhibitory activity, 200 μL of 2.5 mM p-NPG (p-nitrophenyl α-glucopyranoside), dissolved in 0.1 M sodium phosphate buffer (pH 7.0), and 20 μL of 0.1 M sodium phosphate buffer (pH 7.0) were added to 20 μL of the extract in a 96 well plate, and then 20 μL of 0.2 U/mL α-glucosidase enzyme solution was added. After that, the resulting mixture was allowed to react at 37° C. for 15 minutes. Afterwards, the absorbance was measured by using a micro reader at 400 nm, and the inhibitory activity was determined by substituting the value to the formula below in comparison with the control group, which was not treated with the sample and to which distilled water was added, and then expressed as a percentage:

$$Enzyme inhibitory activity ( \% ) = \frac{Control group absorbance - Absorbance with added sample}{Control group absorbance} \times 100$$

TABLE 3

| Concentration | Comparative | |
|---|---|---|
| (%) | Example | Example |
| 0.1 | 41.4 ± 4.3 | 76.6 ± 4.9 |
| 1 | 88.9 ± 0.9 | 96.7 ± 0.4 |
| 10 | 91.3 ± 0.5 | 99.6 ± 0.2 |
| 20 | 92.8 ± 0.5 | 99.7 ± 0.6 |
| 50 | 103 ± 2.3 | 106.2 ± 6.4 |
| 100 | 113.8 ± 4.6 | 114.1 ± 7.7 |

The anti-diabetic CHABATAE beverage composition of the present invention exhibited the highest inhibitory activity of 76.6% at a concentration of 0.1%. In addition, the inhibitory activity was found to be higher than that of the 100% agarwood-fermented green tea extract of Comparative Example, suggesting that the fermented guava leaf and fermented mulberry leaf extract has an α-glucosidase inhibitory activity.

The description of the present invention described above is for illustrative purposes, and those skilled in the art will understand that the present invention can be easily modified into other specific forms without changing the technical idea or essential features of the present invention. Therefore, the embodiments described above should be understood in all respects as illustrative and not restrictive. For example, each component described as a single form may be implemented in a distributed manner, and similarly, components described as distributed may also be implemented in a combined form.

The scope of the present invention is represented by the patent claims described below, and all changes or modified forms derived from the meaning and scope of the claims and their equivalent concepts should be construed as being included in the scope of the present invention.

What is claimed is:

1. An anti-diabetic CHABATAE beverage composition comprising:
mineral water;
a fermented guava leaf extract;
a fermented mulberry leaf extract; and
an agarwood-fermented green tea extract.

2. The anti-diabetic CHABATAE beverage composition according to claim 1, wherein the mineral water includes at least one selected from the group consisting of calcium, potassium, germanium, magnesium, iron, and zinc.

3. The anti-diabetic CHABATAE beverage composition according to claim 1, wherein the mineral water has a pH of 7 to 11.

4. The anti-diabetic CHABATAE beverage composition according to claim 1, wherein the mineral water is 95% to 98% by volume of the anti-diabetic CHABATAE beverage composition.

5. The anti-diabetic CHABATAE beverage composition according to claim 1, wherein the fermented guava leaf extract and the fermented mulberry leaf extract are 0.5% to 3% by volume of the anti-diabetic CHABATAE beverage composition.

6. The anti-diabetic CHABATAE beverage composition according to claim 1, wherein the fermented guava leaf extract is made from early-maturing guava leaves.

7. The anti-diabetic CHABATAE beverage composition according to claim 1, wherein the agarwood-fermented green tea extract is 0.5% to 3% by volume in the anti-diabetic CHABATAE beverage composition.

8. The anti-diabetic CHABATAE beverage composition according to claim 1, further comprising a Jerusalem artichoke-bitter gourd extract,
wherein the Jerusalem artichoke-bitter gourd extract is 0.5% to 3% by volume in the anti-diabetic CHABATAE beverage composition.

9. A method for preparing an anti-diabetic CHABATAE beverage composition, the method comprising:
a step of preparing fermented mulberry leaves and fermented guava leaves by fermenting mulberry leaves and guava leaves;
a step of obtaining a fermented mulberry leaf-fermented guava leaf extract by putting the fermented mulberry leaves, the fermented guava leaves, and mineral water into a non-pressurized extractor and heating the same;
a step of obtaining an agarwood-fermented green tea extract by putting mineral water and agarwood-fermented green tea into a non-pressurized extractor and heating the same; and
a step of obtaining a mixed extract by putting the fermented mulberry leaf-fermented guava leaf extract and the agarwood-fermented green tea extract into heated mineral water and heating the same.

10. The method for preparing an anti-diabetic CHABATAE beverage composition according to claim 9, wherein the step of preparing fermented mulberry leaves and fermented guava leaves is fermenting the mulberry leaves and the guava leaves in a red clay fermentation reactor for 20 to 48 hours at a humidity of 70% to 80% and a temperature of 50° C. to 90° C.

11. The method for preparing an anti-diabetic CHABATAE beverage composition according to claim 9, wherein the step of obtaining a fermented mulberry leaf-fermented guava leaf extract is putting 90% to 95% by weight of the mineral water, 1% to 4% by weight of the fermented mulberry leaves, and 2% to 6% by weight of the fermented guava leaves to the non-pressure extractor and extracting for 1 to 12 hours in a temperature range of 60° C. to 100° C.

12. The method for preparing an anti-diabetic CHABATAE beverage composition according to claim 9, wherein the step of obtaining an agarwood-fermented green tea extract is putting 95% to 98% by weight of the mineral water and 2% to 5% by weight of the agarwood-fermented green tea into the non-pressurized extractor and extracting for 1 to 12 hours in a temperature range of 60° C. to 100° C.

13. The method for preparing an anti-diabetic CHABATAE beverage composition according to claim 9, wherein the step of obtaining a mixed extract is putting 95% to 98% by volume of the heated mineral water, 0.5% to 3% by volume of the fermented mulberry leaf-fermented guava leaf extract, and 0.5% to 3% by volume of the agarwood-fermented green tea extract into the non-pressurized extractor and extracting for 1 to 12 hours in a temperature range of 80° C. to 120° C.

14. The method for preparing an anti-diabetic CHABATAE beverage composition according to claim 9, further comprising a Jerusalem artichoke-bitter gourd extract,
wherein the Jerusalem artichoke-bitter gourd extract is obtained by putting mineral water, Jerusalem artichoke, and bitter gourd into a non-pressurized extractor and heating the same, and
wherein the Jerusalem artichoke-bitter gourd extract is obtained by putting 90% to 95% by weight of the mineral water, 1% to 6% by weight of the Jerusalem artichoke, and 1% to 4% by weight of the bitter gourd into a non-pressurized extractor and extracting for 1 to 12 hours in a temperature range of 60° C. to 100° C.

15. The method for preparing an anti-diabetic CHABATAE beverage composition according to claim 9, further comprising, after the step of obtaining a mixed extract, a step of sterilizing the mixed extract for 5 to 60 minutes in a temperature range of 90° C. to 130° C.
